(19) United States
(12) Patent Application Publication
(10) Pub. No.: US 2022/0111115 A1
(43) Pub. Date: Apr. 14, 2022

(54) A BONE CEMENT COMPOSITION

(71) Applicants: AGENCY FOR SCIENCE, TECHNOLOGY AND RESEARCH, Singapore (SG); NATIONAL UNIVERSITY OF SINGAPORE, Singapore (SG)

(72) Inventors: Yi Yan YANG, Singapore (SG); Zhen Chang LIANG, Singapore (SG); Chuan YANG, Singapore (SG); Ee Jen Wilson WANG, Singapore (SG)

(73) Assignees: AGENCY FOR SCIENCE, TECHNOLOGY AND RESEARCH, Singapore (SG); NATIONAL UNIVERSITY OF SINGAPORE, Singapore (SG)

(21) Appl. No.: 17/264,608

(22) PCT Filed: Jul. 30, 2019
(86) PCT No.: PCT/SG2019/050379

§ 371 Date: Jan. 29, 2021

(30) Foreign Application Priority Data
Jul. 30, 2018 (SG) 10201806501W

(51) Int. Cl. A61L 24/06; C08G 64/02; C08G 64/18; C08G 64/40; A61L 24/00

(52) U.S. Cl. CPC A61L 24/06; C08G 64/02; C08G 64/18; A61L 2300/406; A61L 24/0015; A61L 2430/02; C08G 64/40

(57) ABSTRACT

There is provided a bone cement composition comprising: a powder component comprising at least one acrylic polymer a liquid component comprising a monomer; an antibiotic; and an acid-functionalised polymer, wherein reaction of the powder component and the liquid component results in formation of a bone cement. In a preferred embodiment, the acid-functionalised polymer is selected from polyethylene glycol-polycarbonate (PEG-PAC), polycarbonate-poly(L-lactide) (PAC-PLLA), polycarbonate-poly(D-lactide) (PAC-PDLA), PAC-PLLA/PDLA, copolymers thereof or a combination thereof. There is also provided a bone cement formed from the bone cement composition.

### TECHNICAL FIELD

The present invention relates to a bone cement composition, a bone cement formed from the bone cement composition and a kit for forming the bone cement.

### BACKGROUND

Implants are extensively used in orthopaedics for arthroplasties and fracture fixations. Due to their excellent biocompatibility and mechanical properties, titanium and its alloys have been developed into key implant materials. Despite good outcomes, disastrous complications such as prosthetic joint infections still remain. Routine treatments, including intravenous antibiotics and surgical debridement, often yield poor results; with revision surgeries still the standard. However, this poses significant morbidity and mortality to patients with risks of surgical and anaesthetic complications.

Prosthetic joint infections arise from a multitude of factors, from patient-related ones, surgical factors such as sterility/allogenic blood transfusions to post-operative factors such as urinary tract infections and prolonged hospitalizations. Prosthetic infections result from the adhesion of bacterial cells to the implant surfaces, leading to aggregation and subsequent bacteria proliferation, which forms an adherent biofilm. Biofilm resistance to antibiotics results in highly recalcitrant infections. The primary prevention of prosthetic infections is thus crucial.

Currently, antibiotic-loaded bone cement is used during implant construct. However, problems in the art with such antibiotic-loaded bone cement is poor antibiotic elution, and a short duration of elution of the antibiotics, often limited to two weeks, after which antibiotic activity is then permanently lost.

There is therefore a need for an improved bone cement for use in orthopaedics.

### SUMMARY OF THE INVENTION

The present invention seeks to address these problems, and/or to provide an improved bone cement for use in orthopaedics, preferably with improved and prolonged antibiotic elution, whilst maintaining mechanical strength adequate for implant fixation.

According to a first aspect, the present invention provides a bone cement composition comprising:

a powder component comprising at least one acrylic polymer;

a liquid component comprising a monomer;

an antibiotic; and

an acid-functionalised polymer,

wherein reaction of the powder component and the liquid component results in formation of a bone cement.

The acid-functionalised polymer may be any suitable polymer. For example, the acid-functionalised polymer may be an acid-functionalised copolymer. According to a particular aspect, the acid-functionalised polymer may be an acid-functionalised block copolymer comprising at least two homopolymer subunits. In particular, one of the at least two homopolymer subunits may be polycarbonate (PAC). Even more in particular, the acid-functionalised block copolymer may be an acid-functionalised diblock copolymer.

Examples of suitable acid-functionalised polymers may be, but not limited to: polyethylene glycol-polycarbonate (PEG-PAC), polycarbonate-poly(L-lactide) (PAC-PLLA), polycarbonate-poly(D-lactide) (PAC-PDLA), PAC-PLLA/PDLA, copolymers thereof or a combination thereof.

The bone cement composition may comprise a suitable amount of the acid-functionalised polymer. For example, the composition may comprise 0.5-15 wt. % of acid-functionalised polymer, based on the total weight of the composition.

The antibiotic comprised in the bone cement composition may be any suitable antibiotic. For example, the antibiotic may be an amine-containing antibiotic. In particular, the antibiotic may be an aminoglycoside antibiotic. Examples of suitable antibiotics may be, but not limited to: amikacin, apramycin, geneticin, gentamicin, kanamycin, netilmicin, neomycin, paromomycin, spectinomycin, streptomycin, tobramycin, polymyxins, or a combination thereof.

According to a second aspect, the present invention provides a bone cement formed from the bone cement composition of the first aspect.

In particular, amine group comprised in the antibiotic and carboxylic group comprised in the acid-functionalised polymer may form a non-covalent bond. Accordingly, the bone cement may exhibit antibacterial activity for ≥100 days. The bone cement may exhibit an area of inhibition zone for bacteria of ≥1 cm² after 100 days. The bone cement may have a compressive modulus of ≥875 MPa.

According to a third aspect, the present invention provides a kit for forming a bone cement, the kit comprising:

a powder component comprising at least one acrylic polymer;

a liquid component comprising a monomer;

an antibiotic; and

an acid-functionalised polymer.

According to a particular aspect, the powder component may further comprise a polymerization initiator.

According to another particular aspect, the liquid component may further comprise a polymerization activator.

### BRIEF DESCRIPTION OF THE DRAWINGS

In order that the invention may be fully understood and readily put into practical effect there shall now be described by way of non-limitative example only exemplary embodiments, the description being with reference to the accompanying illustrative drawings. In the drawings:

FIG. 1 shows the synthesis of diblock copolymer of PEG and carboxylic acid-functionalized polycarbonate (PEG-PAC);

FIG. 2 shows the synthesis of PAC-PLLA and PAC-PDLA via organocatalytic ring-opening polymerization of carboxylic acid-functionalized cyclic carbonate and lactide;

FIG. 3 shows the effect of polymer doping on setting times on gentamicin, gentamicin/PEG and gentamicin/PEG-PAC loaded cements;

FIGS. 4a and 4b show the area of inhibition zones for S. aureus and P. aeruginosa respectively, when using various bone cement formulations (plain cement without gentamicin, cement with gentamicin, cement with gentamicin and PEG or PEG-PAC at various concentrations);

FIGS. 5a and 5b show the area of inhibition zones for S. aureus and P. aeruginosa respectively, when using various bone cement formulations (plain cement without gentamicin, cement with gentamicin, cement with PEG, cement with gentamicin and PAC-PDLA, PAC-PLLA or PAC-PDLA/PLLA at various concentrations);

FIGS. 6a to 6c respectively show the effect of polymer doping on compressive modulus, compressive yield and compressive strength (15% strain) of bone cement;

FIGS. 7a to 7c respectively show the effect of vacuum on compressive modulus, o compressive yield and compressive strength (15% strain) of bone cement, with polymer content at 1%; and

FIG. 8 shows the effect of polymer doping on alkaline phosphatase (ALP) synthesis activity.

### DETAILED DESCRIPTION

As explained above, there is a need for an improved bone cement for use in orthopaedics, preferably with improved and prolonged antibiotic elution, whilst maintaining mechanical strength adequate for implant fixation.

In general terms, the invention relates to a bone cement which provides improved elution kinetics of antibiotics. The bone cement of the present invention provides effective and sustained release of the antibiotic loaded in the bone cement. In particular, acid-functionalised polymers may be doped into the antibiotic-loaded bone cement to prolong antibacterial function of the antibiotic and to increase antibacterial efficacy without compromising on the mechanical properties of the bone cement. Even more in particular, the acid-functionalised polymers added to the antibiotic-loaded bone cement form complexes with the antibiotic which thereby prolongs the release of the antibiotic and increases the antibacterial efficacy.

According to a first aspect, the present invention provides a bone cement composition comprising:

a powder component comprising at least one acrylic polymer;

a liquid component comprising a monomer;

an antibiotic; and

an acid-functionalised polymer,

wherein reaction of the powder component and the liquid component results in formation of a bone cement.

The acid-functionalised polymer may be any suitable polymer. In particular, the acid-functionalised polymer may be a biodegradable polymer. For the purposes of the present invention, an acid-functionalised polymer may be defined as any polymer which is functionalised by the chemical attachment of a carboxylic group to its structure. The acid-functionalised polymer may be formed from any suitable method.

According to a particular aspect, the acid-functionalised polymer may be an acid-functionalised copolymer. The acid-functionalised copolymer may be derived from one or more species of monomer. In particular, the acid-functionalised copolymer may comprise polycarbonate (PAC).

According to another particular aspect, the acid-functionalised polymer may be an acid-functionalised block copolymer. For the purposes of the present invention, the acid-functionalised block copolymer may be defined as a block copolymer comprising two or more homopolymers forming ‘blocks’ of repeating units. At least one of the two or more homopolymers may be acid-functionalised and comprise a free carboxylic acid group.

For example, the acid-functionalised block copolymer may comprise at least two homopolymer subunits, wherein one of the homopolymer subunits is acid-functionalised. In particular, one of the at least two homopolymer subunits may be PAC. Even more in particular, the acid-functionalised block copolymer may be an acid-functionalised diblock copolymer.

Examples of suitable acid-functionalised polymers may be, but not limited to: carboxylic acid-functionalised polycarbonate, diblock copolymer of polyethylene glycol and carboxylic acid-functionalised polycarbonate (PEG-PAC), diblock copolymer of carboxylic acid-functionalised polycarbonate and poly(L-lactide) (PAC-PLLA), diblock copolymer of carboxylic acid-functionalised polycarbonate and poly(D-lactide) (PAC-PDLA), benzoic acid-functionalised polycarbonate, diblock copolymer of PEG and benzoic acid-functionalised polycarbonate, diblock copolymer of benzoic acid-functionalised polycarbonate and PLLA, diblock copolymer of benzoic acid-functionalised polycarbonate and PDLA, sulfonic acid-functionalised polycarbonate, diblock copolymer of PEG and sulfonic acid-functionalised polycarbonate, diblock copolymer of sulfonic acid-functionalised polycarbonate and PLLA, diblock copolymer of sulfonic acid-functionalised polycarbonate and PDLA, phosphate-functionalised polycarbonate, diblock copolymer of PEG and phosphate-functionalised polycarbonate, diblock copolymer of phosphate-functionalised polycarbonate and PLLA, diblock copolymer of phosphate-functionalised polycarbonate and PDLA, PAC-PLLA/PDLA, copolymers thereof or a combination thereof.

The bone cement composition may comprise a suitable amount of the acid-functionalised polymer. For example, the composition may comprise 0.5-15 wt. % of acid-functionalised polymer, based on the total weight of the composition. In particular, the composition may comprise 1-10 wt. % of acid-functionalised polymer, based on the total weight of the composition. Even more in particular, the composition may comprise 1 wt. % or 5 wt. % of acid-functionalised polymer, based on the total weight of the composition.

The antibiotic comprised in the bone cement composition may be any suitable antibiotic. The antibiotic may be effective in reducing, inhibiting, and/or preventing the growth or transmission of foreign organisms in a patient.

The antibiotic may be provided in any form in which the antibiotic has antibiotic efficacy or which enables the release of a compound having an antibiotic effect. Therefore, for the purposes of the present invention, antibiotic may be defined as encompassing antibiotic salts or antibiotic esters, as well as the corresponding hydrated forms of the antibiotic, antibiotic salts or antibiotic esters.

According to a particular aspect, the antibiotic may be a water-soluble antibiotic. The antibiotic may be an amine-containing antibiotic. In particular, the antibiotic may be an aminoglycoside antibiotic. For example, the antibiotic may be, but not limited to: amikacin, apramycin, geneticin, gentamicin, kanamycin, netilmicin, neomycin, paromomycin, spectinomycin, streptomycin, tobramycin, polymyxins, or a combination thereof. In particular, the antibiotic may be gentamicin or a gentamicin derivative including, but not limited to, gentamicin salts or gentamicin esters. For example, the gentamicin salt may be gentamicin sulphate. The gentamicin sulphate may be a mixture of the gentamicin homologs, C1a, C1, C2a, C2b, and C2. The advantage of gentamicin is that it does not crystallise due to the presence of a mixture of multiple gentamicin homologs. Further, gentamicin may tolerate elevated temperatures for brief periods of time without loss of its antimicrobial efficacy.

The bone cement composition may comprise a suitable amount of the antibiotic. For example, the composition may comprise 0.5 wt. % to 15 wt. % of antibiotic, based on the total weight of the composition. In particular, the composition may comprise 1 wt. % or 5 wt. % of antibiotic, based on the total weight of the composition.

According to a particular aspect, the powder component may be any suitable powder component known in the art for bone cement. For example, the powder component may comprise an acrylic polymer. The acrylic polymer may be any acrylic polymer suitable for use in bone cement. In particular, the acrylic polymer may be homopolymers or copolymers of acrylic acid esters, methacrylic acid esters, styrene, vinyl derivatives or their mixtures. The powder component may also comprise a suitable polymerization initiator. The initiator may be any suitable initiator for the purposes of the present invention.

The powder component may also comprise X-ray contrast media, such as, for example, zirconium dioxide, dyestuffs for identification, such as, for example, chlorophyll, and fillers, and if appropriate other additives. Customary additives are, for example, calcium phosphates which have an osteoinductive or osteoconductive action, such as, in particular, hydroxy-apatite and tricalcium phosphate. The content of all these additives can vary within a relatively wide range and depends on the particular profile of requirements of the bone cement or of the corresponding secondary products.

The liquid component comprised in the bone cement composition may be a reactive liquid monomer component that polymerizes about the powder component. For example, the reactive liquid may contain reactive organic monomers selected from methylmethacrylate, homolog esters of methacrylic acid or their mixtures. The liquid component may also comprise a polymerization accelerator, such as dimethyl-p-toluidine, and hydroquinone as a stabilizer in amounts customary for these. Dyestuffs and other expedient additives may furthermore be present.

The preparation of bone cement generally involves mixing the powder and liquid components in a suitable reaction vessel or mixing receptacle to form the bone cement. Generally, it is necessary that the components of bone cement be uniformly and thoroughly mixed so that a homogenous bone cement may be obtained. Increased homogeneity of the bone cement may be particularly desirable in providing a bone cement mixture that is easy to work with, yet maintains satisfactory mechanical properties. In producing bone cement, it is typical to maintain the liquid and the powder components separate until just prior to use and to avoid exposure of the components to the atmosphere because of the potentially irritating and flammable nature of the bone cement components.

According to a particular aspect, the bone cement may be formed from the bone cement composition by mixing the acid-functionalised polymer with the liquid component and the antibiotic, followed by mixing the resultant mixture with the powder component to initiate polymerization and formation of the bone cement.

The bone cement formed from the bone cement composition may have improved antibacterial properties. In particular, the bone cement formed exhibits effective and sustained release of antibiotic comprised in the bone cement. For example, the antibacterial function of the bone cement may be prolonged and the antibacterial efficacy may be significantly increased without compromising the mechanical properties of the cement.

According to a second aspect, the present invention provides a bone cement formed from the bone cement composition described above.

The bone cement may be useful in the prevention and treatment of bacterial infections caused during bone transplantation. In particular, the bone cement of the present invention enables prolonged antibacterial function of antibiotics and increases antibacterial efficacy without compromising its mechanical properties. This is due to the doping of the bone cement with acid-functionalised polymers. In particular, the acid-functionalised polymers may be as described above in relation to the bone cement composition.

According to a particular aspect, amine group of the antibiotic comprised in the bone cement and carboxylic group of the acid-functionalised polymer comprised in the bone cement may form a non-covalent bond. In particular, the antibiotic and the acid-functionalised polymer comprised in the bone cement may form a complex through ionic interaction between the amine group in the antibiotic and the carboxylic group in the acid-functionalised polymer.

The formation of the complex between the antibiotic and the acid-functionalised polycarbonate through electronic interaction allows for sustained release of the antibiotic, hence prolonging antimicrobial activity of the cement.

The bone cement according to the present invention may exhibit improved antibacterial activity. For example, the bone cement may have antibacterial activity for ≥100 days. In particular, the bone cement may have antibacterial activity for >250 days against S. aureus, and >160 days against P. aeruginosa.

The bone cement may exhibit an improved area of inhibition zone for bacteria. For the purposes of the present invention, inhibition zone is defined as the area around the bone cement where there is no growth of bacteria. In particular, the inhibition zone for bacteria may be ≥1 cm2 after 100 days.

The bone cement of the present invention also has good mechanical integrity and this is not compromised as a result of the improved antibacterial properties of the bone cement. For example, the bone cement may have a compressive modulus of ≥875 MPa.

The present invention also provides a kit for forming a bone cement, the kit comprising:

a powder component comprising at least one acrylic polymer;

a liquid component comprising a monomer;

an antibiotic; and

an acid-functionalised polymer.

The powder component, liquid component, antibiotic and acid-functionalised polymer may be as described above.

According to a particular aspect, the powder component may further comprise a polymerization initiator. The polymerization initiator may be any suitable polymerisation initiator.

According to another particular aspect, the liquid component may further comprise a polymerization activator. The polymerization activator may be any suitable polymerisation activator.

The kit may further comprise instructions on the use of the kit.

Having now generally described the invention, the same will be more readily understood through reference to the following embodiment which is provided by way of illustration, and is not intended to be limiting.

### EXAMPLE

### Materials

All chemicals were purchased from Sigma-Aldrich and utilized as received unless otherwise indicated. All solvents were of analytical grade, purchased from Fisher Scientific and used as received. The cyclic carbonate benzyl, 1-methyl-4-oxocyclohexane-1-carboxylate (MTC-OBn) was prepared according to theprotocol as cited in Pratt et. al., Chemical Communications, 2008, Vol 1: 114-116. Polyethylene glycol (PEG) (Methoxy PEG (MPEG), number average molecular weight (Mn) 9,870 Da, polydispersity index (PDI) 1.02) was bought from Rapp Polymere (Tuebingen, Germany).

### Nuclear Magnetic Resonance (NMR) Spectroscopy

The ¹H NMR spectra of monomers and polymers were recorded on a Bruker Advance 400 NMR spectrometer at 400 MHz at room temperature. The ¹H NMR measurement parameters: acquisition time of 3.2 seconds, pulse repetition time of 2.0 seconds, 30° C. pulse width, 5208-Hz spectral width, and 32 K data points. Chemical shifts were referred to the solvent peak (δ=2.50 and 7.26 ppm for DMSO-d₆ and CDCl₃, respectively).

### Polymer Synthesis

### (a) Synthesis of PEG-PAC

MTC-OBn (0.27 g, 1.08 mmol) and MPEG (0.59 g, 0.06 mmol) were dissolved in CH₂Cl₂ (3 mL), and 1,8-Diazabicyclo[5.4.0]undec-7-ene (DBU) (24 μL, 0.15 mmol) was added to the above solution to initiate the polymerization under stirring. After reacted for 3 hours, benzoic acid (about 25 mg) was added to quench the polymerization. The reaction mixture was then precipitated into diethyl ether (40 mL), and the precipitate was centrifuged and washed with diethyl ether (30 mL) for three times before dried in vacuo, to give PEG-P(MTC-OBn) as white powder (0.70 g, 86%). ¹H NMR (400 MHz, CDCl₃, 22° C.): δ 7.29 (m, 75H, PhH), 5.12 (s, 30H, —OCH₂Ph), 4.27 (m, 60H, —CH₂OCOO), 3.63 (m, 897H, H of MPEG), 1.22 (s, 30H, —CH₃). PDI: 1.08.

Deprotection of benzyl groups: A mixture of the above polymers (0.7 g), tetrahydrofuran (THF) (7.5 mL), methanol (7.5 mL), and Pd-C (10% w/w, 0.2 g) was swirled under H₂ (7 atm) overnight. After evacuation of the H₂ atmosphere, the mixture was filtered by syringe. Additional mixture of THF and methanol (1:1 in volume, 7.5 mL) were used to ensure complete transfer. The collected washings were evaporated, and the residue was precipitated into diethyl ether (40 mL). Then, the precipitate was centrifuged, washed with diethyl ether (30 mL) for three times, and dried in vacuo, to give the final product PEG-PAC as white powder. The yield was more than 90%, and ¹H NMR spectra showed that the protected groups were removed after hydrogenation.

### (b) Synthesis of PAC-PLLA/PDLA

Ring-opening polymerization (ROP) of PAC-PLLA: MTC-OBn (0.5 g, 2 mmol) and 4-methylbenzyl alcohol (4-MBA, 12.4 mg, 0.1 mmol) were dissolved in CH₂Cl₂ (2 mL), and DBU (15 μL, 0.1 mmol) was added to the above solution to initiate the polymerization under stirring. After reacted for 1 hour, the solution of L-lactide (0.173 g, 1.2 mmol) in CH₂Cl₂ (1 mL) was added to the above reaction mixture and the reaction continued for another 1.5 hour before quenched by benzoic acid (about 15 mg). The reaction solution was then purified by column chromatography on a Sephadex LH-20 column with THF as eluent, to give P(MTC-OBn)-PLLA as white powder (0.54 g, 80%). ¹H NMR (400 MHz, CDCl₃, 22° C.): δ 7.30 (m, 115H, PhH), 5.13 (s, 71H, —OCH₂Ph of MTC-OBn and —OC(O)CHO— of PLLA), 4.27 (m, 92H, —CH₂OCOO), 2.34 (s, 3H, —CH₃ of 4-MBA), 1.58 (d, 75H, —CH₃ of PLLA), 1.22 (s, 69H, —CH₃ of MTC-OBn). PDI: 1.19.

The removal of protecting benzyl groups of P(MTC-OBn)-PLLA was similar to that of PEG-PAC, and the final PAC-PLLA was obtained in more than 90% yield. In this way, PAC-PDLA was also synthesized in high yield. PDI: 1.18.

### Gel Permeation Chromatography (GPC)

GPC analysis for polymers was carried out on a GPC system (Waters 2690, MA, U.S.A.) with an Optilab rEX differential refractometer detector (Wyatt Technology Corporation, U.S.A.) and Waters HR-4E column. The mobile phase used was THF with a flow rate of 1 mL/min. Number-average molecular weights and polydispersity indices were calculated from a calibration curve obtained using a series of polystyrene standards (Polymer Laboratories Inc., MA, U.S.A., with molecular weight ranging from 1,350 to 151,700).

### Preparation of Bone Cement Samples

Plain and gentamicin-loaded high viscosity cement were commercially procured from Johnson & Johnson (SmartSet: Depuy CMW, Blackpool, UK) and used for the purposes of this example. The various polymers were synthesized according to the protocols as stated above and subsequently doped into bone cement samples. Briefly, the synthesized polymers were first completely dissolved in the cement's liquid monomer (1% and 5% w/w), before being mixed with the powder component at the manufacturer's recommended mixing ratio (1:2). Mixing was carried out using a ceramic bowl and polymeric spatula under ambient conditions (room temperature and atmospheric pressure). This cement dough was subsequently extruded into cylindrical moulds (12 mm length: 6 mm diameter) and allowed to set for 20 minutes. Cement samples were visually inspected for surface defects. Acceptable samples were polished with sandpaper and used for subsequent experiments as detailed below.

### Doughing Time

Doughing time of bone cement specimens were determined in accordance with established procedures as detailed in ASTM F451-08. Mixed cement samples were probed at 15-second intervals using a non-powdered latex-gloved finger 1 minute after the onset of mixing. The time at which cement separated cleanly from the gloved finger on probing was taken as the doughing time of the respective cement sample.

### Bacterial Culture

The two representative strains of bacteria commonly encountered in orthopaedic infections that were used in this study were: 1) gram-positive Stapylococcus aureus and 2) gram-negative Pseudomonas aeruginosa. Each bacterial strain was cultured in BBL™ Mueller Hinton Broth (MHB, HD Singapore) at 37° C. All microorganisms were cultured overnight for 24 hours to reach mid-logarithmic growth phase. The concentration of microbes used for experiments as detailed below were adjusted by means of optical density (OD) readings. An OD reading of 0.07 at 600 nm translates to the concentration of Mc Farland 1 solution (3×10⁸ CFU/ml) on a microplate reader (TECAN, Switzerland).

### Inhibition Zone Studies

10 μl of bacteria was inoculated on Lsyogeny broth (LB) agar plates using a cell spreader. The bone cement specimens were placed in the middle of these bacteria-innoculated agar plates and incubated at 37° C. to simulate physiological conditions. The diameters (D) of inhibition zones were then measured at pre-determined intervals: Day 1, 3, 5, 7 and then weekly thereafter until zones were completely absent. Inhibition zones were expressed in terms of their area (A) using the formula A=π(D/2).

### Compressive Modulus/Yield/Compressive Strength

The compressive mechanical properties of polymer-doped cement constructs were measured to determine the effect of polymer doping on the bone cement's mechanical performance. In accordance with ISO 5833, 6 mm×12 mm cylindrical constructs were compressed at a cross-head speed of 20 mm/min at a failure load of 40% using a mechanical testing machine (MTS 858 Bionix Universal Testing Machine). The compressive modulus was calculated as the slope of the initial linear portion of the stress-strain curve. The compressive yield was determined by the stress measured at the point of permanent yield on the stress-strain curve. The compressive strength was measured by dividing the maximum load by the cross-sectional area of the bone cement specimen.

### ALP Synthesis of Osteoblasts

Osteoblasts were seeded into at a density of 5000 cells/cm² and cultured in growth media with 50 μg/ml ascorbic acid and 10 mM sodium β-glycerophosphate. Bone cement formulations at various concentrations were then added into trans-wells for a duration of 2 weeks. The wells were then subsequently washed using phosphate-buffered saline (PBS) and cells lysed after 3 repeated cycles of freezing and thawing. The amount of p-nitrophenol released was used to determine ALP activity. Briefly, 100 μl of cell lysate was added to 100 μl of p-nitrophenol substrate (Sigma) and subjected to incubation at 37° C. for 30 minutes, before 50 μl of 1M NaOH was added to terminate the reaction. OD was then measured using a microplate reader at 405 nm. The amount of p-nitrophenol was then quantified using a standard curve from known concentrations of p-nitrophenol.

### Results

### Polymer Synthesis

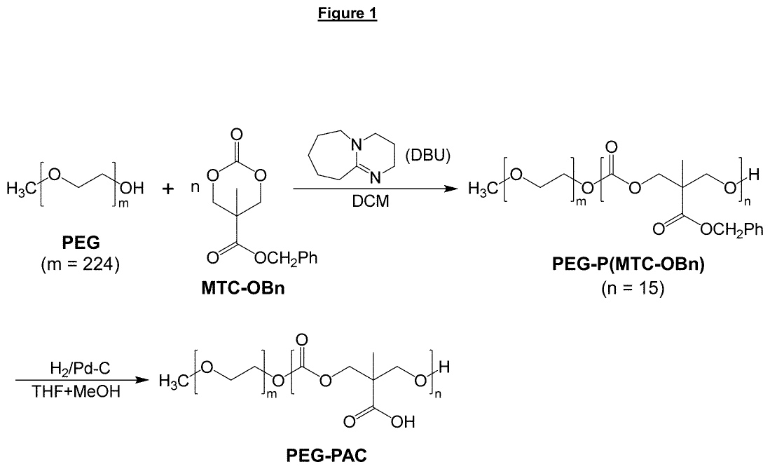

A diblock copolymer of PEG (Mn 9,870 kDa, PDI 1.02) and carboxylic acid-functionalized polycarbonate (PAC, 15 degree of polymerization) was synthesized through a highly controlled metal-free organocatalytic ring-opening polymerization method (OROP) (FIG. 1). For this living polymerization, PEG as a macroinitiator initiated the polymerization of cyclic carbonate monomer, MTC-OBn, in the presence of DBU as a catalyst. The reaction was conducted in dichloromethane (DCM) at room temperature for 3 hours before being quenched with benzoic acid and isolated by precipitation in diethyl ether. The composition of the diblock copolymer PEG-P(MTC-OBn) was assessed by 1H NMR and the molar ratio of PEG and MTC-OBn in the polymer matched that in the feed well. There were 15 units of MTC-OBn in a polymer, which was estimated by comparing the integral intensities of the peak of the methyl protons of MTC-OBn at 1.22 ppm and those of ethylene hydrogens of PEG at 3.64 ppm. The polymer was subjected to hydrogenolysis at 7 atmosphere of H₂ gas to remove the benzyl protecting groups, giving the final product PEG-PAC in high yield. ¹H NMR result showed the peaks attributing to the benzyl groups disappeared, indicating a complete conversion of MTC-OBn to MTC-OH (with a free carboxylic acid group). PAC was designed to interact with gentamicin through ionic/electrostatic interaction for sustained release, and the hydrophilic nature of both PEG and PAC blocks created water channels, promoting drug elution.

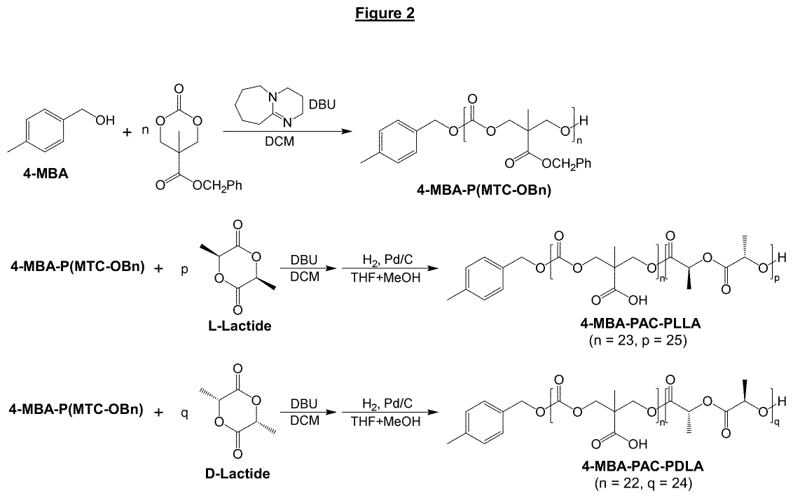

To further improve mechanical strength, PEG was replaced with a hydrophobic block in the polymer. Specifically, carboxylic acid-functionalized polycarbonate/poly(L-lactide) diblock copolymer (PAC-PLLA) and carboxylic acid-functionalized polycarbonate/poly(D-lactide) diblock copolymer (PAC-PDLA) were synthesized by OROP (FIG. 2). For this living polymerization, the block polymers were synthesized by sequential monomer addition of MTC-OBn and L-type or D-type Lactide in the presence of 4-methylbenzyl alcohol (4-MBA) as an initiator and DBU as a catalyst. The reaction was conducted for 2.5 hours before being quenched by benzoic acid and the polymer P(MTC-OBn)-PLLA/PDLA was purified via size exclusion chromatography. The compositions of the diblock copolymer P(MTC-OBn)-PLLA/PDLA was quantitatively estimated by ¹H NMR measurements. By comparison of the integral intensities of the peaks of the respective methyl protons of the initiator 4-MBA at 2.34 ppm, MTC-OBn at 1.22 ppm, and L-lactide at 1.58 ppm, the number of MTC-OBn units and L-Lactide (L-LA) in P(MTC-OBn)-PLLA was 23 and 25, respectively. Similarly, the number of MTC-OBn units and D-Lactide (D-LA) in P(MTC-OBn)-PDLA was 22 and 24, respectively. Then, both polymers were subjected to hydrogenolysis at 7 atmosphere of H₂ gas to remove the benzyl protecting groups, giving the final product PAC-PLLA or PAC-PDLA in high yield, respectively. Formation of stereocomplexation between PLLA and PDLA was envisioned to compensate the loss of mechanical strength, which was caused by PEG.

### Effect of Polymer Doping on Cement Setting Time

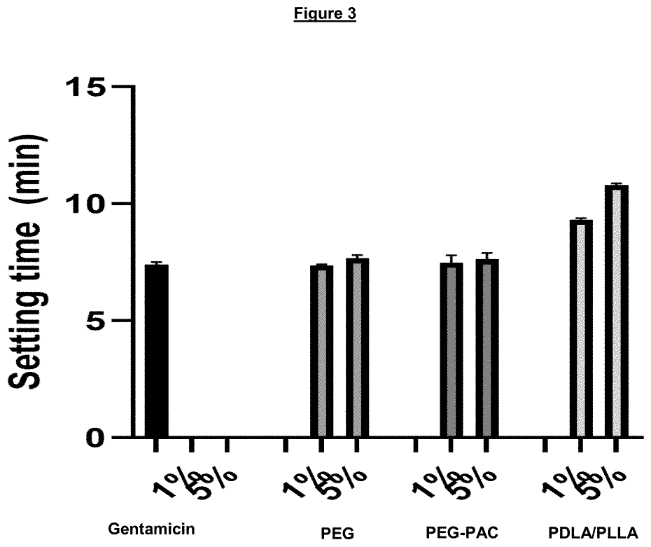

The results demonstrated that the doping of commercial bone cement with PAC-PLLA/PDLA significantly increased cement setting times compared to un-doped cement. This prolongation of setting times was unique for PAC-PDLA/PLLA. At 1% concentration, the setting times of PAC-PLLA/PDLA-doped cement was 9.32±0.07 minutes. When polymer concentration was increased to 5%, setting time was further increased to 10.8±0.06 minutes. In contrast, the setting time of undoped gentamicin cement was measured at 7.40±0.10 minutes. The rest of the polymers had comparable setting times to undoped cement as shown in FIG. 3.

Cement doughing and setting time is particularly relevant in the clinical setting as it determines the amount of time the operating surgeon has to optimally position their cemented implants to achieve the best outcomes for their patients. Doughing and setting times in both extremes (too short or too long) can potentially lead to suboptimal outcomes and devastating complications for affected patients. Improperly-positioned implants when setting times are too short can result in complications such as implant loosening and poor clinical outcomes e.g. reduction/limitation in range of joint motion, increased post-operative pain. Unreasonably long setting times in extreme cases will unduly prolong operative times. This compounds both anaesthetic and surgical risks for patients, potentially resulting in the increasing likelihood of post-operative infection and intra-operative complications such as strokes/cardiac events and even death. However, when doughing/setting times are optimally prolonged (within reasonable limits), surgeons are afforded the added advantage of longer working times. This allows them greater flexibility time-wise to focus on the critical step of implant cementation—the crux of arthroplasty procedures which when improperly done, can lead to devastating complications as afore-mentioned.

### Effect of Polymer Doping on Inhibition Zones

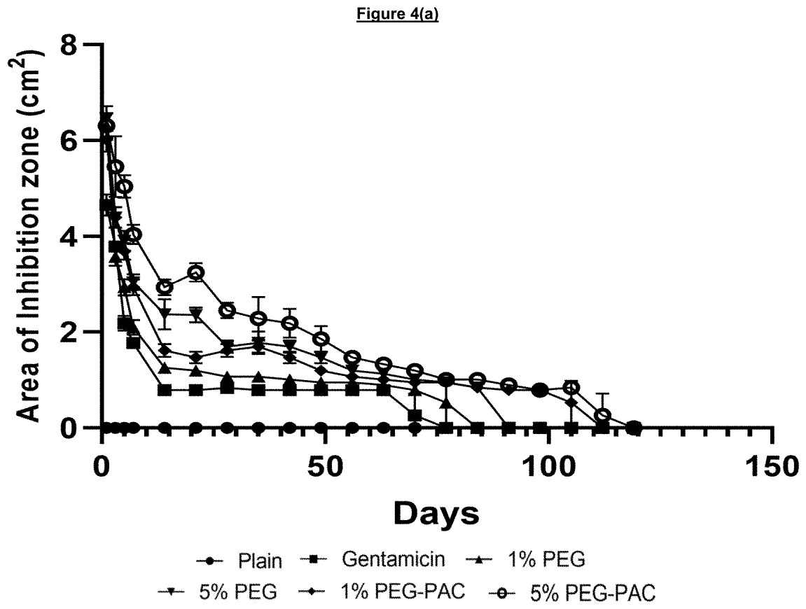

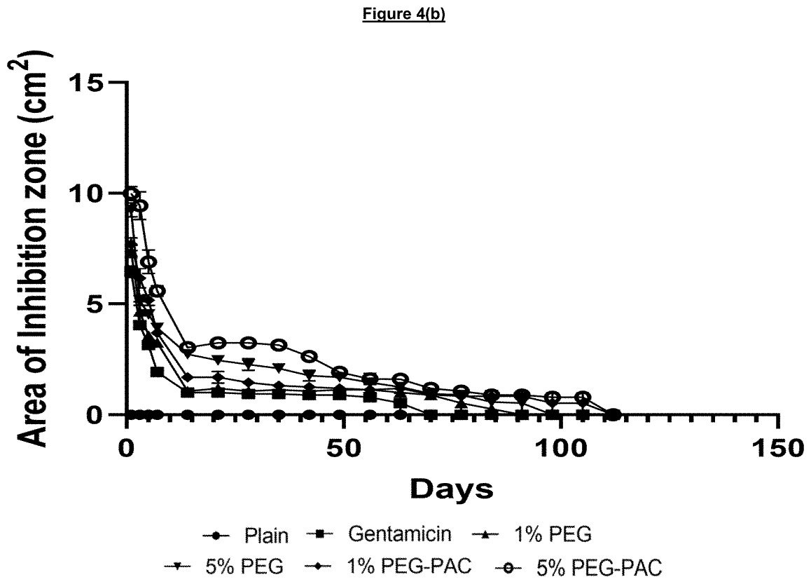

The diblock copolymer was mixed with commercially available gentamicin-loaded bone cement Depuy SmartSet at various concentrations (1% and 5%) in comparison with PEG. Inhibition zone studies were performed to evaluate antibacterial performance of polymer-loaded cement. As shown in FIG. 4, the addition of both PEG and PEG-PAC significantly increased the inhibition zones for both S. aureus (FIG. 4a) and P. aeruginosa (FIG. 4b) compared to the negative controls. Even up till Day 42 post-incubation, the inhibition zones of 5% PEG-PAC were still at least twice as large compared to undoped controls (both S. aureus and P. aeruginosa). Compared to PEG, PEG-PAC inhibitions were also observed to be consistently larger, reaching up to 1.5 times against both S. aureus and P. aeruginosa. This enhanced antibacterial effect may be due to the increased wettability introduced by the combination of PEG and PAC compared to PEG alone, resulting in increased gentamicin elution from porous cement channels.

Besides larger inhibition zones, PEG-PAC also demonstrated prolonged antibacterial activity of bone cement compared to PEG and negative controls. Inhibition zones persisted for 119 days (S. aureus) and 112 days (P. aeruginosa) with 5% PEG-PAC, compared to 91 (S. aureus) and 98 days (P. aeruginosa) with 5% PEG. This represents a prolongation of between 2-4 weeks. This was due to the acid component of PEG-PAC binding with the NH₂ component of gentamicin, resulting in sustained release of the antibiotic.

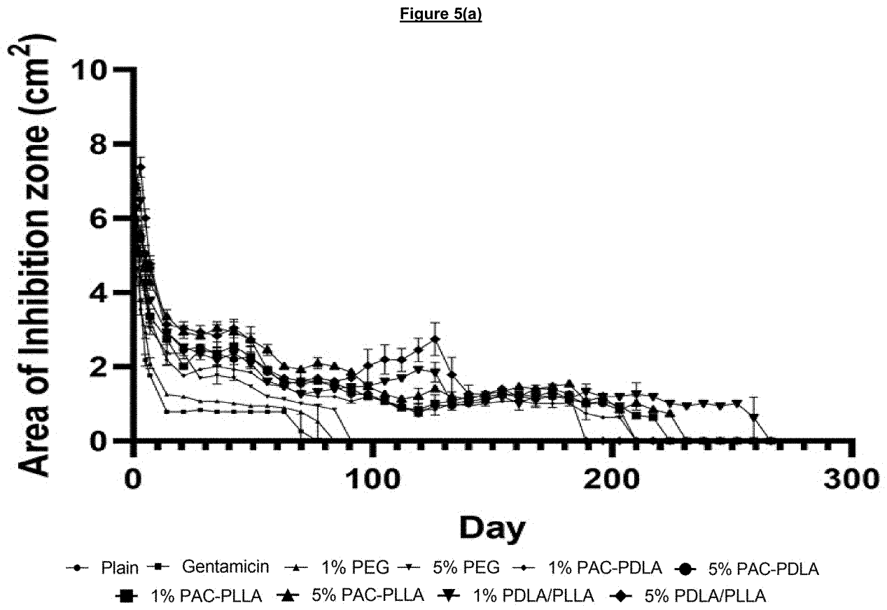

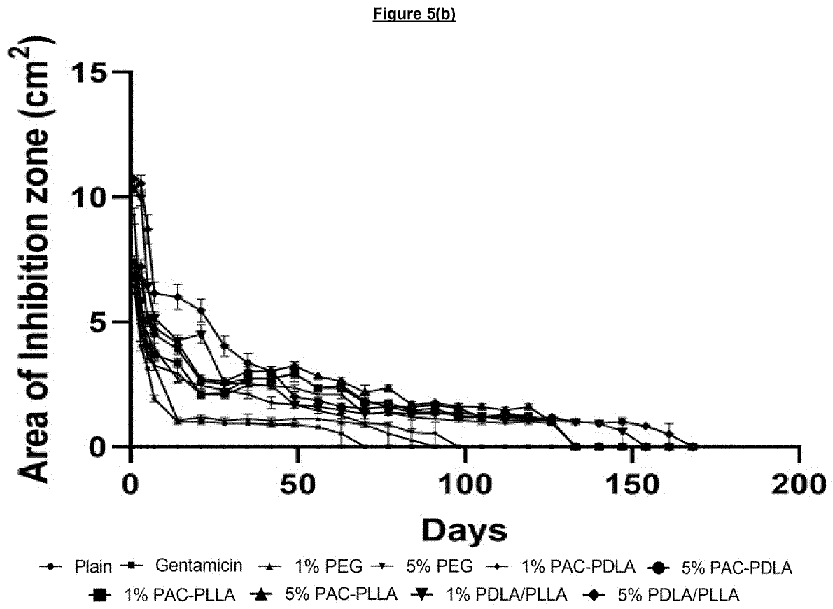

The addition of PAC-PDLA, PAC-PLLA and PAC-PDLA/PLLA further improved upon the good antibacterial performance already achieved by the PEG-PAC polymers. At Day 91 post-incubation, the inhibition zones of our best performing polymer, 5% PAC-PLLA cement was more than 2 times that of 5% PEG-PAC cement. This was observed consistently against both representative bacterial strains S. aureus and P. aeruginosa. The stereo-complexed combination of PAC-PDLA/PLLA initially exhibited the best elution kinetics against both S. aureus and P. aeruginosa (as denoted by largest inhibition zones), and the antibacterial effect of PAC-PDLA/PLLA stereo-complex last for a longer period of time, i.e. more than 259 days and 147 days against S. aureus and P. aeruginosa, respectively (FIG. 5). This indicates that stereo-complexes achieved more sustained drug release.

Increasing the stereo-complex content to 5% led to larger inhibition zones and longer antibacterial functional period against P. aeruginosa (161 days vs. 147 days) (FIG. 5b).

### Effect of Polymer Doping on Mechanical Properties of Bone Cement

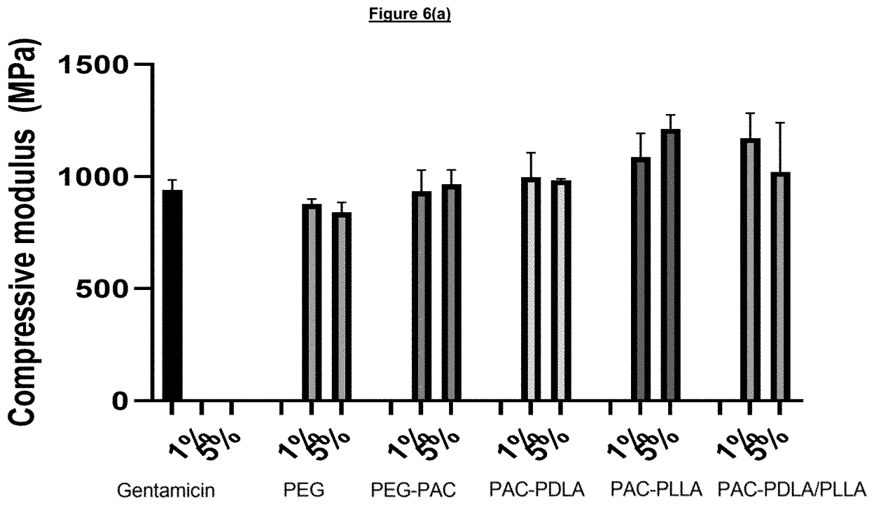

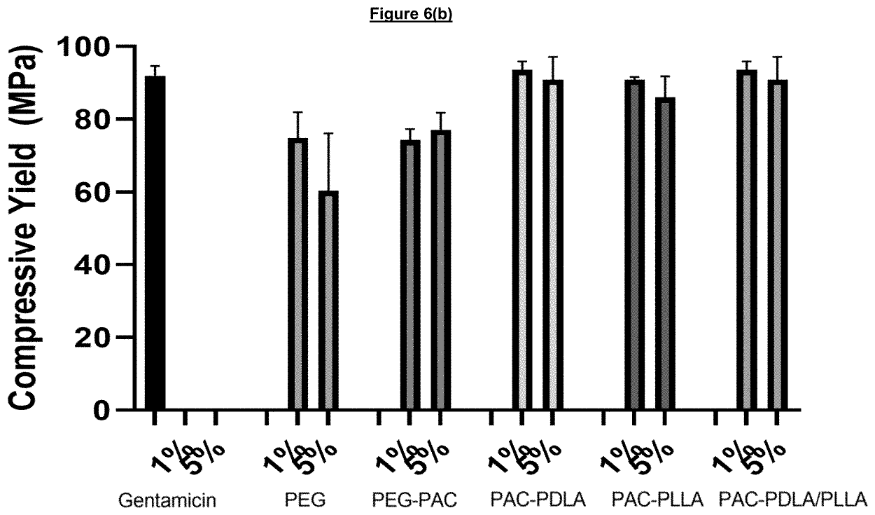

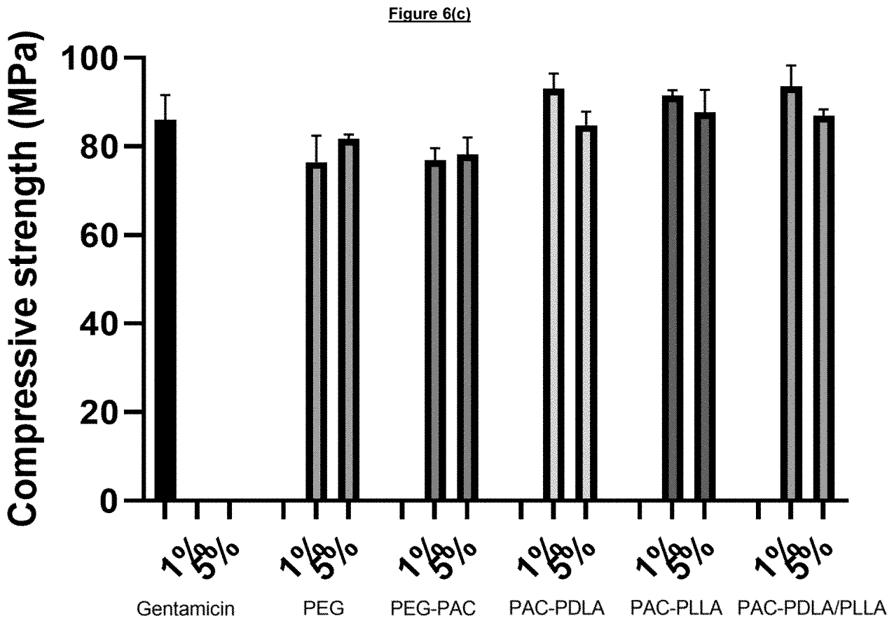

The addition of 5% PEG significantly reduced the bone cement's mechanical strength compared to the negative undoped controls. This was seen consistently across all 3 different strength parameters: compression modulus, compressive yield and compressive strength (FIGS. 6a to 6c). However, when bone cement was doped with other polymers instead, the bone cement's mechanical strength was preserved (FIGS. 6b and 6c). The presence of the stereo-complex increased compressive modulus especially at 1% (FIG. 6a).

The compressive modulus, compressive yield and compressive strength of the bone cement were preserved after adding the acrylic acid monomers especially at 1%.

### Effect of Vacuum on Mechanical Properties of Bone Cement

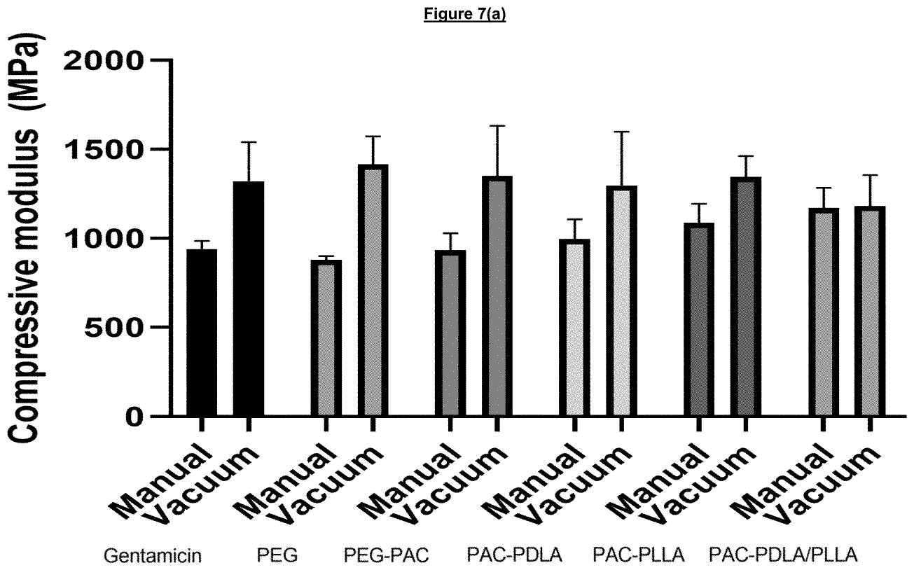

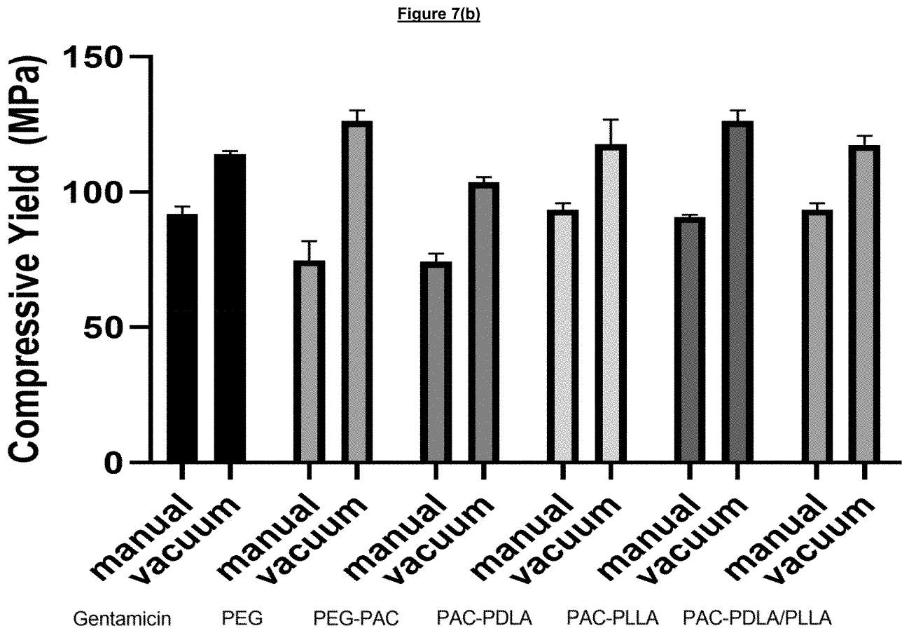

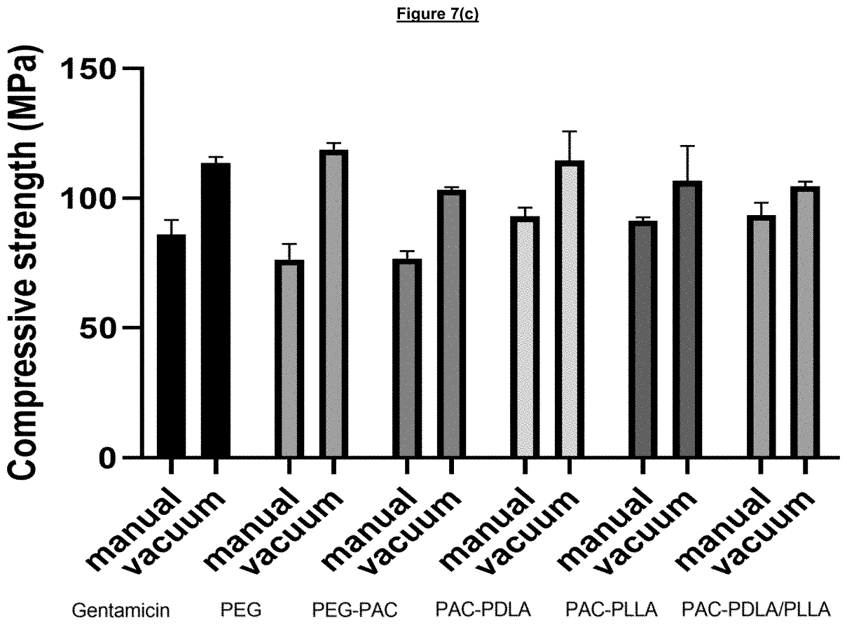

Under vacuum, cements formed had significantly higher mechanical strength, as vacuum can reduce the formation of bubbles during mixing, resulting in lower porosity. This was seen consistently across all 3 different strength parameters: compression modulus, compressive yield and compressive strength (FIGS. 7a to 7c), for undoped cement and for cements doped with 1% polymer.

### Effect of Polymer Doping on ALP Synthesis of Osteoblasts

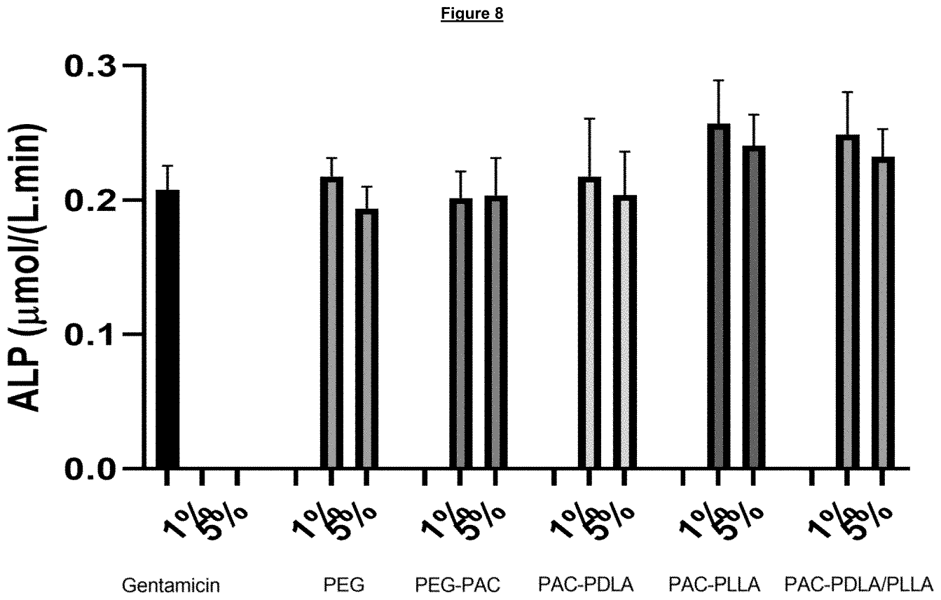

ALP level indicates osteoblast activity. As shown in FIG. 8, polymer doping at both 1% and 5% did not affect ALP synthesis of osteoblasts significantly, indicating polymer doping did not cause significant cytotoxicity in osteoblasts.

Whilst the foregoing description has described exemplary embodiments, it will be understood by those skilled in the technology concerned that many variations may be made without departing from the present invention.

1. A bone cement composition comprising:
a powder component comprising at least one acrylic polymer;
a liquid component comprising a monomer;
an antibiotic; and
an acid-functionalised polymer,
wherein reaction of the powder component and the liquid component results in formation of a bone cement.

2. The bone cement composition according to claim 1, wherein the acid-functionalised polymer is an acid-functionalised copolymer.

3. The bone cement composition according to claim 1, wherein the acid-functionalised polymer is an acid-functionalised block copolymer comprising at least two homopolymer subunits.

4. The bone cement composition according to claim 3, wherein one of the at least two homopolymer subunits is polycarbonate (PAC).

5. The bone cement composition according to claim 3, wherein the acid-functionalised block copolymer is an acid-functionalised diblock copolymer.

6. The bone cement composition according to claim 1, wherein the acid-functionalised polymer is: polyethylene glycol-polycarbonate (PEG-PAC), polycarbonate-poly(L-lactide) (PAC-PLLA), polycarbonate-poly(D-lactide) (PAC-PDLA), PAC-PLLA/PDLA, copolymers thereof or a combination thereof.

7. The bone cement composition according to claim 1, wherein the composition comprises 0.5-15 wt. % of acid-functionalised polymer, based on total weight of the composition.

8. The bone cement composition according to claim 1, wherein the antibiotic is an amine-containing antibiotic.

9. The bone cement composition according to any preceding claim 1, wherein the antibiotic is an aminoglycoside antibiotic.

10. The bone cement composition according to claim 9, wherein the aminoglycoside antibiotic is: amikacin, apramycin, geneticin, gentamicin, kanamycin, netilmicin, neomycin, paromomycin, spectinomycin, streptomycin, tobramycin, polymyxins, or a combination thereof.

11. A bone cement formed from the bone cement composition of claim 1.

12. The bone cement according to claim 11, wherein amine group comprised in the antibiotic and carboxylic group comprised in the acid-functionalised polymer form a non-covalent bond.

13. The bone cement according to claim 11, wherein the bone cement exhibits antibacterial activity for ≥100 days.

14. The bone cement according to claim 11, wherein the bone cement exhibits an area of inhibition zone for bacteria of ≥1 cm² after 100 days.

15. The bone cement according to claim 11, wherein the bone cement has a compressive modulus of ≥875 MPa.

16. A kit for forming a bone cement, the kit comprising;
a powder component comprising at least one acrylic polymer;
a liquid component comprising a monomer;
an antibiotic; and
an acid-functionalised polymer.

17. The kit according to claim 16, wherein the powder component further comprises a polymerization initiator.

18. The kit according to claim 16, wherein the liquid component further comprises a polymerization activator.

19. The bone cement composition according to claim 3, wherein the acid-functionalised block copolymer is an acid-functionalised diblock copolymer and wherein one of the at least two homopolymer subunits is polycarbonate (PAC).
